(19) United States
(12) Patent Application Publication
(10) Pub. No.: US 2025/0295294 A1
(43) Pub. Date: Sep. 25, 2025

(54) STEERABLE AND ROTATABLE MEDICAL DEVICE

(71) Applicant: Boston Scientific Scimed, Inc., Maple Grove, MN (US)

(72) Inventor: Jonathan MELCHOR, Providence, RI (US)

(73) Assignee: Boston Scientific Scimed, Inc., Maple Grove, MN (US)

(21) Appl. No.: 19/082,521

(22) Filed: Mar. 18, 2025

Related U.S. Application Data
(60) Provisional application No. 63/567,229, filed on Mar. 19, 2024.

(51) Int. Cl. A61B 1/005; A61B 1/00; A61B 90/00

(52) U.S. Cl. CPC A61B 1/0052; A61B 1/00042; A61B 1/00128; A61B 90/03; A61B 2090/034

(57) ABSTRACT

Medical devices and related methods useful for facilitating the rotation of a medical device to multiple planes are described. The medical device may include a handle that comprises a proximal portion and a distal portion coupled to the proximal portion by a connector configured to be gripped by a user. The distal portion may be rotatable relative to the proximal portion by rotation of the connector and an articulation mechanism may be disposed within the proximal portion and the distal portion of the handle and extend through the connector. The articulation mechanism may be configured to control articulation of a shaft coupled to the handle and includes a coupler translatable relative to the connector independent of rotating the connector.

### CROSS-REFERENCE TO RELATED APPLICATIONS

This application claims the benefit of priority to U.S. Provisional Application No. 63/567,229, filed on Mar. 19, 2024, which is incorporated by reference herein in its entirety.

### TECHNICAL FIELD

Aspects of the present disclosure relate generally to medical devices and actuator mechanisms for medical devices. More specifically, the present disclosure relates to medical systems and devices for selectively adjusting an orientation of a medical device along various planes.

### BACKGROUND

During a medical procedure, a user may operate a medical device that includes a handle and a shaft extending distally therefrom. Some devices also incorporate a steering mechanism, which allows the user to deflect the distal tip of the shaft of the device. However, to alter the deflection of the distal tip in multiple planes, the user may have to rotate handle of the device in an uncomfortable manner, leading to increasing fatigue over time and difficulties while maneuvering the device.

For example, medical professionals may experience wrist and hand discomfort from manually manipulating the medical device to reach different planes. As a result, the procedure may be prolonged and/or introduce risks to the patient.

### SUMMARY

The present disclosure includes medical devices that facilitate the rotation of a medical device to multiple planes.

For example, the present disclosure includes a handle that comprises a proximal portion and a distal portion coupled to the proximal portion by a connector configured to be gripped by a user. The distal portion may be rotatable relative to the proximal portion by rotation of the connector and an articulation mechanism may be disposed within the proximal portion and the distal portion of the handle and extend through the connector. The articulation mechanism may be configured to control articulation of a shaft coupled to the handle and includes a coupler translatable relative to the connector independent of rotating the connector.

According to some aspects, a shaft or a proximal end of the shaft may be coupled to a distal end of the distal portion of the handle and the articulation mechanism may include at least one articulation member that extends from the handle through the shaft. The proximal portion of the handle includes an actuator, and user input received by the actuator controls translation of the coupler. The connector may include a knob. The coupler may include a proximal extension that abuts a distal extension and is rotatable relative to the distal extension. The coupler may comprise a proximal slider that includes the proximal extension and a distal slider that may include the distal extension. The proximal portion of the handle may include an actuator and user input received by the actuator may control translation of the coupler. The distal slider may be configured to rotate by rotation of the connector without rotating the proximal slider. The connector may be biased in a direction towards the proximal portion of the handle. The proximal portion of the handle may include a spring that biases the connector towards the proximal portion of the handle. The proximal portion of the handle may include a locking mechanism to lock a rotational position of the connector relative to the proximal portion of the handle. Additionally, the connector may define a plurality of apertures and the locking mechanism may include a pin receivable into each aperture. The articulation mechanism may include a cam coupled to at least one articulation member; the cam being contained within the distal portion of the handle. Further, for example, the cam may be coupled to the coupler and configured to rotate by translation of the coupler.

The present disclosure also includes a medical device comprising a handle having a proximal portion that includes an actuator and a distal portion coupled to the proximal portion by a connector configured to be gripped by a user. The distal portion may be rotatable relative to the proximal portion by rotation of the connector. A shaft may be coupled to the distal portion of the handle and an articulation mechanism may be configured to control articulation of the shaft. The articulation mechanism may be disposed within the handle and extend through the connector. The articulation mechanism may include a coupler translatable relative to the connector independent of rotating the connector. For example, the connector may include a proximal slider coupled to the actuator and a distal slider coupled to at least one articulation member that extends from the handle through the shaft. Further, the coupler may be translatable relative to the connector independent of rotating the connector.

According to some aspects, a distal end of the proximal slider abuts a proximal end of the distal slider, the distal slider being rotatable relative to the proximal slider. The coupler may include a housing that surrounds the distal end of the proximal slider and the proximal end of the distal slider. The handle may include a locking mechanism to restrict rotation of the distal portion of the handle relative to the proximal portion of the handle. In some example, the locking mechanism may include a pin insertable into a plurality of apertures defined by the connector.

The present disclosure also includes a medical device comprising a handle having a proximal portion and a distal portion coupled to the proximal portion by a connector configured to be gripped by a user. The distal portion may be rotatable relative to the proximal portion by rotation of the connector and an articulation mechanism that may be disposed within the proximal portion and the distal portion of the handle and extending through the connector. The articulation mechanism may be configured to control articulation of a shaft coupled to the handle and may include a coupler translatable relative to the connector independent of rotating the connector. According to some aspects, the proximal portion of the handle may include a pin insertable into a plurality of apertures defined by the connector to selectively restrict rotation of the distal portion of the handle relative to the proximal portion of the handle.

### BRIEF DESCRIPTION OF THE FIGURES

The accompanying drawings, which are incorporated in and constitute a, illustrate exemplary aspects that, together with the written descriptions, serve to explain the principles of this disclosure. Each figure depicts one or more exemplary aspects according to this disclosure, as follows:

FIG. 1 depicts a front plan cut-away view of an exemplary medical device according to some aspects of the present disclosure.

FIG. 2 depicts the proximal handle portion of the medical device of FIG. 1.

FIG. 3 depicts the proximal handle portion, the connector of the medical device, and a distal portion of the medical device of FIG. 1.

FIG. 4 depicts a front plan cut-away view of the medical device of FIG. 1 while rotating the handle.

FIG. 5A depicts the connector of the medical device of FIG. 1.

FIG. 5B depicts features of the distal portion of the handle of the medical device of FIG. 1.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Aspects of the present disclosure are now described with reference to exemplary medical devices, systems, and methods useful for facilitating navigation of a medical device in an ergonomic fashion for the user. For example, the medical device may include a handle configured to rotate the shaft of the medical device without rotating in the entire handle, e.g., by rotating a distal portion of the handle of the medical device relative to a proximal portion of the handle. The proximal portion and the distal portion of the handle may be coupled together by a rotatable connector. The medical device may include an articulation mechanism disposed within the handle and configured to control the shaft of the medical device along one or more planes. The articulation mechanism may extend through the proximal portion of the handle, the connector, and the distal portion of the handle. The connector may be configured to be gripped by a user to permit selective rotation of the connector and the distal portion of the handle relative to the proximal portion of the handle. The articulation mechanism may include features that allow a user to deflect the distal tip of the shaft of the medical device and rotate the shaft independent of the deflection.

Reference will now be made in detail to examples to help illustrate aspects of the present disclosure through the accompanying drawings. Wherever possible, the same reference numbers will be used throughout the drawings to refer to the same or like parts.

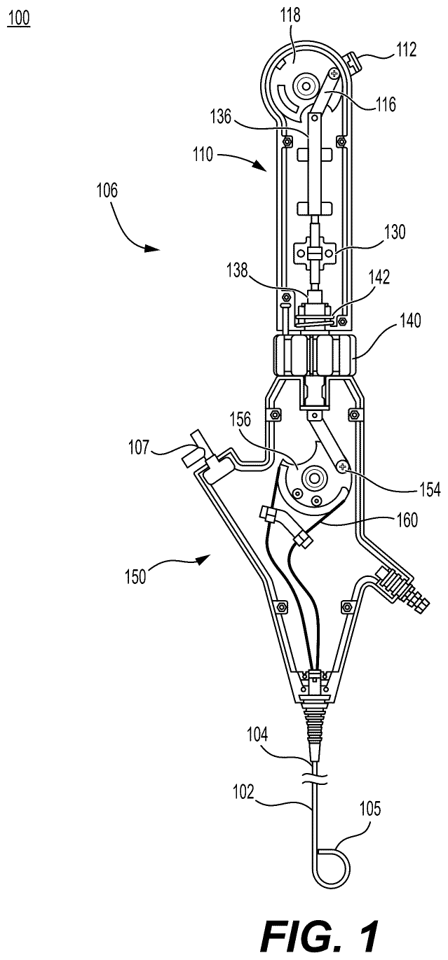

FIG. 1 illustrates an exemplary medical device 100, including a handle 106 and a shaft 102. The medical device 100 may be any suitable device configured to allow a user to access and view internal areas of a subject's body and perform medical diagnoses and/or treatments on a subject. For example, the medical device 100 may be a ureteroscope, an endoscope, a hysteroscope, a bronchoscope, a cystoscope, or other scope or similar medical device.

The handle 106 may include a port 107 in communication with a working channel defined by the shaft 102. Auxiliary instruments, such as, for example, biopsy forceps, graspers, baskets, snares, and/or other devices may be inserted into the port 107 and advanced through the working channel to exit through a distal opening of the distal end 105 of the shaft 102 into a subject's body to perform a medical procedure. The shaft 102 may provide access into a bodily lumen (e.g., along a tortuous path, for example, via a natural body orifice).

The handle 106 includes a proximal portion 110 and a distal portion 150 coupled together by a connector 140. The connector 140 may be rotatable and allow for rotational movement of the distal portion 150 relative to the proximal portion 110 of the handle. While the connector 140 is illustrated as a knob, other shapes are contemplated herein as long as the connector is rotatable. The handle 106 may have any suitable shape able to be gripped by a user's hand. For example, the proximal portion 110 of the handle 106 may include a grip portion proximal to the connector 140. As further discussed below, the connector 140 likewise may be configured to be gripped by a user. The distal portion 150 of the handle may be coupled to a proximal end 104 of the shaft 102. The proximal end 104 of the shaft may be detachably or permanently connected to a distal end of the distal portion 150 of the handle 106.

The handle 106 also includes an actuator 112 operably coupled to an articulation mechanism to control movement of the shaft 102. In this example, the port 107 is shown on the distal portion 150 of the handle 106 while the actuator 112 is shown on the proximal portion 110 of the handle 106, however other arrangements are contemplated herein. The actuator 112 is shown in the figures as a rotatable lever but may be configured as another type of actuator such as a knob, switch, or thumbwheel. As will be discussed in more detail below, the actuator 112 may be operably coupled (e.g., directly or indirectly) to an articulation mechanism in the handle 106 and one or more articulation members 160 (e.g., pull wire(s)) extending through the handle 106 and/or the shaft 102.

The articulation mechanism includes a coupler 130 translatable relative to the connector 140, e.g., along a longitudinal axis of the handle 106. In some examples, the coupler 130 includes a proximal extension that abuts a distal extension, the distal extension being rotatable relative to the proximal extension. For example, the coupler 130 may include a proximal slider 136 and a distal slider 138 coupled together and rotatable relative to each other.

The actuator 112 may control translation of the coupler 130 in order to employ one or more articulation members 160 to deflect the distal end 105 of the shaft 102. The actuator 112 may be operably coupled to a connecting rod 116 and a cam 118, in turn operably coupled to the proximal slider 136, in order to transfer motion of the actuator 112 (e.g., in the case of the actuator 112 being a lever, rotation of the lever via user input) to translation along a longitudinal axis of the medical device 100. Similarly, the distal slider 138 may be coupled to a connecting rod 154 and a cam 156 to drive rotation of the cam 156 to thereby control tension applied to one or more articulation members 160 (two articulation members 160 being shown in FIG. 1) coupled to the cam 156. The articulation member(s) 160 may extend through the shaft 102 (or otherwise be operably coupled to articulation member(s) extending through the shaft 102) to deflect the shaft 102 relative to the longitudinal axis of the handle 106 and of the medical device 100.

Once the distal end 105 of the shaft 102 is in a desired position relative to the longitudinal axis of the medical device 100 (e.g., deflected away from the longitudinal axis), the user may adjust the plane in which the shaft 102 extends by rotating the shaft 102. To rotate the shaft 102 without rotating the hand that grips the handle 106 and controls the actuator 112, the user may grip the proximal portion 110 of the handle 106 with one hand while rotating the connector 140 (e.g., using the other hand) in order to rotate the distal portion 150 of the handle 106 and the shaft 102 relative to the proximal portion 110 of the handle 106. Because the distal portion 150 is rotatable relative to the proximal portion 110, the user need not adjust their grip on the proximal portion 110 to rotate the shaft 102.

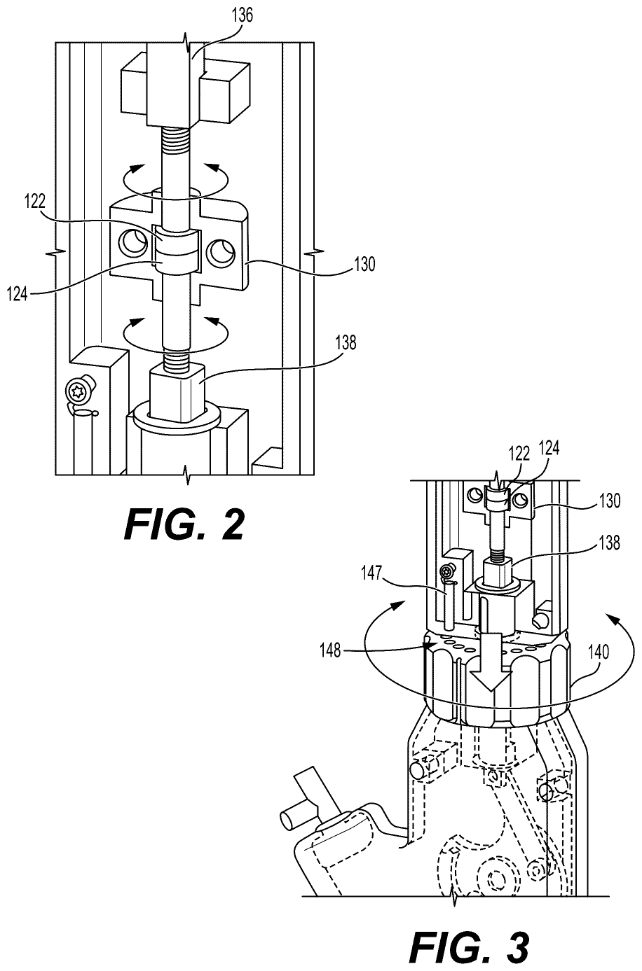

FIG. 2 shows an exploded view of features of the articulation mechanism, including the proximal slider 136 and the distal slider 138 of the coupler 130. The proximal slider 136 and at least a part of the distal slider 138 may be within the proximal portion 110 of the handle 106. As mentioned above, the proximal slider 136 may be coupled to the cam 118 and the connecting rod 116. The connection may include, for example, a mechanical fastener (e.g., a screw, a setscrew, a bolt, a rivet, etc.) or adhesive. The handle 106 may include features to guide movement of the proximal slider 136 and distal slider 138 in a linear direction.

The coupler 130 may include a housing that at least partially or completely surrounds a distal end 122 of the proximal slider 136 and a proximal end 124 of the distal slider 138. In some examples, the proximal slider 136 and the distal slider 138 have surfaces that abut each other, e.g., within the housing, and that permit rotation of the proximal slider 136 relative to the distal slider 138. In some examples, as illustrated in the figures, the distal end of the proximal slider 136 and the proximal end of the distal slider 138 are each cylindrical in shape with flat surfaces that abut each other. Other shapes are contemplated herein, provided that the proximal slider 136 is rotatable relative to the distal slider 138. For example, the housing of the coupler 130 may define a cavity with sufficient room to permit rotation of distal slider 138 relative to proximal slider 136. While FIG. 2 shows the housing has defining a generally cylindrical cavity, this is exemplary only and other shapes are contemplated herein.

The proximal slider 136 may be a single integrated piece or may include two or more components coupled together. For example, the proximal slider 136 may include an extension that includes the distal end 122 of the proximal slider 136, the extension being integral with or fixedly coupled to the proximal end of the proximal slider 136 (the proximal end of the proximal slider 136 being coupled to the connecting rod 116, cam 118, and actuator 112). While FIG. 2 shows a threaded connection that fixes the extension to the remainder of the proximal slider 136, this is exemplary only. Other types of mating elements or an adhesive may be used, or the extension may be integral with the remainder of the proximal slider 136.

Similarly, the distal slider 138 may be a single integrated piece or may include two or more components coupled together. For example, the distal slider 138 may include an extension that includes the proximal end 124 of the distal slider 138, the extension being integral with or fixedly coupled to the distal end of the distal slider 138 (the distal end of the distal slider 138 being coupled to the connecting rod 154, cam 156, and articulation members 160). While FIG. 2 shows a threaded connection that fixes the extension to the remainder of the distal slider 138, this is exemplary only. Other types of mating elements or an adhesive may be used, or the extension may be integral with the remainder of the distal slider 138.

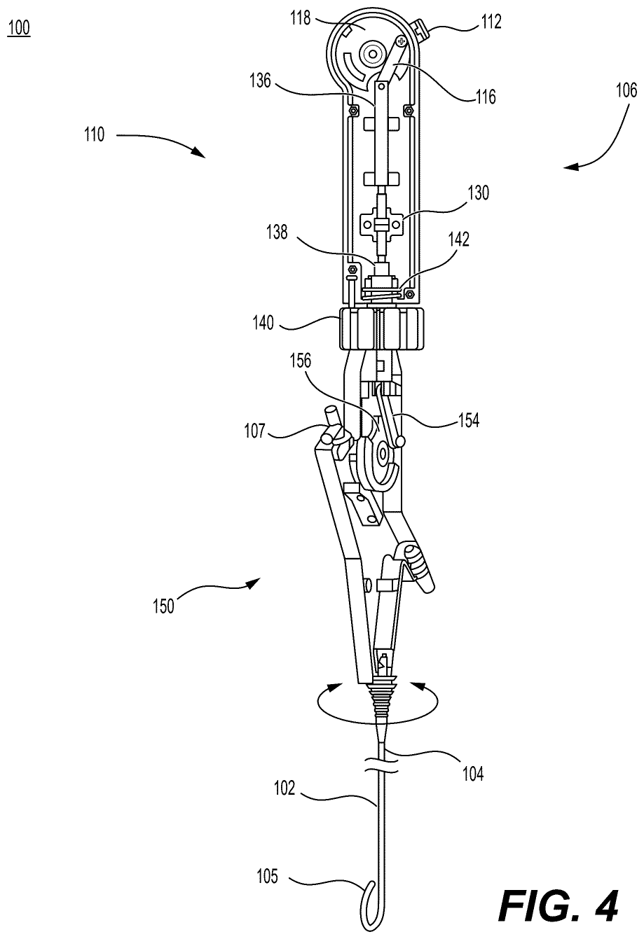

The distal slider 138 may have a geometric cross-section that inhibits rotation of the distal slider 138 relative to the connector 140 and relative to the distal portion 150 of the handle 106. In this way, rotation of the connector 140 may cause a corresponding rotation of the distal slider 138 and the distal portion 150 of the handle 106. In aspects of the present disclosure, at least a portion of the distal slider 138 may have a rectangular (e.g., square) cross-sectional shape that is slidably received within a corresponding rectangular (e.g., square) aperture of the connector 140. Thus, the distal slider 138 may be translatable within the handle 106, relative to the connector 140, without being rotatable relative to the connector 140. This configuration permits a user to rotate the connector 140 in order to rotate the distal portion 150 of the handle 106 without engaging the articulation member(s) 160 coupled to the distal slider 138. FIG. 4 illustrates the medical device 100 of FIG. 1 after rotation of the distal portion 150 of the handle 106 and without changing the deflection of the distal end 105 of the shaft 102.

As discussed above, deflection of the shaft 102 may be achieved via user input at the actuator 112. The user engages the actuator 112, which in turn controls translation of the coupler 130 along a longitudinal axis of the handle 106 (and along a longitudinal axis of the medical device 100). Rotation of the actuator 112 may engage the cam 118 within the proximal portion 110 of the handle 106, which transmits motion via the connecting rod 116 to the proximal slider 136 of the coupler 130. Because the distal end 122 of the proximal slider 136 abuts the proximal end 124 of the distal slider 138, translation of the proximal slider 136 causes translation of the distal slider 138. The translation of the distal slider 138 causes the connecting rod 154 to rotate the cam 156 and control tension of the articulation member(s) 160 coupled to the cam 156 in order to deflect the shaft 102.

According to some aspects of the present disclosure, the handle 106 includes a locking mechanism in order to restrict rotation of the distal portion 150 relative to the proximal portion 110. An exemplary locking mechanism is illustrated in FIG. 3. The locking mechanism may include a pin 147 receivable into one of a plurality of apertures 148. For example, the proximal portion 110 of the handle 106 may include the pin 147 and the connector may include the plurality of apertures 148. The apertures 148 may be spaced apart at regulator intervals, such as evenly spaced at ten-degree intervals. The spacing between the apertures 148 (and thus the total number of apertures 148) shown in the drawings is merely exemplary and non-limiting of other examples (e.g., apertures spaced at 45-degree intervals, 30-degree intervals, etc.).

The connector 140 may be biased in a proximal direction, e.g., via a spring 142. In order to rotate the connector 140, a user may apply force in a distal direction to overcome the biasing force of the spring 142 and release the pin 147 from a corresponding aperture 148. The pin 147 may be retracted from its position within one of the apertures 148 to unlock the locking mechanism, enabling free rotation of the connector 140 with the distal portion 150 of the handle 106. Once the pin 147 is released, the connector 140 may be permitted to rotate relative to the proximal portion 110 of the handle 106.

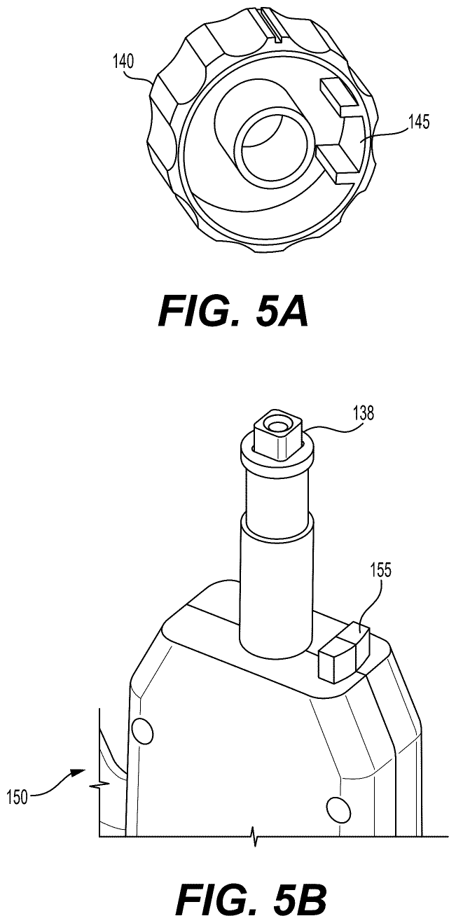

FIGS. 5A and 5B depict mating elements of the connector 140 and the distal portion 150 of the handle 106 that permit the connector 140 to cause corresponding rotation of the distal portion 150 of the handle 106 (e.g., when a user rotates the connector 140). For example, the connector 140 includes a slot or cavity 145 that receives a corresponding projection 155 of the distal portion 150. The shapes of the cavity 145 and projection 155 may permit translation of the connector 140 relative to the distal portion 150 of the handle 106 to facilitate engaging the locking mechanism as discussed above.

The connector 140 may have surface features to facilitate a user's grip on the connector 140, such as ribs or beveled features. When the user has rotated the shaft 102 to face a desired direction via rotation of the distal portion 150 of the handle 106 coupled to the shaft 102, the pin 147 may be reinserted into a corresponding one of the apertures 148 to lock the medical device 100 in the desired position. The user may release the connector 140 to cause the connector 140 to move proximally due to the biasing force of the spring 142 and lock the position of the handle 106 by the pin 147 being inserted into the corresponding aperture 148.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a handle having a proximal portion and a distal portion coupled to the proximal portion by a connector configured to be gripped by a user, wherein the distal portion is rotatable relative to the proximal portion by rotation of the connector; and
an articulation mechanism disposed within the proximal portion and the distal portion of the handle and extending through the connector, wherein the articulation mechanism is configured to control articulation of a shaft coupled to the handle and includes a coupler translatable relative to the connector independent of rotating the connector.

2. The medical device of claim 1, further comprising the shaft, wherein a proximal end of the shaft is coupled to a distal end of the distal portion of the handle, and wherein the articulation mechanism includes at least one articulation member that extends from the handle through the shaft.

3. The medical device of claim 2, wherein the proximal portion of the handle includes an actuator, and user input received by the actuator controls translation of the coupler.

4. The medical device of claim 1, wherein the connector includes a knob.

5. The medical device of claim 1, wherein the coupler includes a proximal extension that abuts a distal extension and is rotatable relative to the distal extension.

6. The medical device of claim 5, wherein the coupler comprises a proximal slider that includes the proximal extension and a distal slider that includes the distal extension.

7. The medical device of claim 6, wherein the proximal portion of the handle includes an actuator, and user input received by the actuator controls translation of the coupler.

8. The medical device of claim 6, wherein the distal slider is configured to rotate by rotation of the connector without rotating the proximal slider.

9. The medical device of claim 1, wherein the connector is biased in a direction towards the proximal portion of the handle.

10. The medical device of claim 9, wherein the proximal portion of the handle includes a spring that biases the connector towards the proximal portion of the handle.

11. The medical device of claim 1, wherein the proximal portion of the handle includes a locking mechanism to lock a rotational position of the connector relative to the proximal portion of the handle.

12. The medical device of claim 11, wherein the connector defines a plurality of apertures, and the locking mechanism includes a pin receivable into each aperture.

13. The medical device of claim 1, wherein the articulation mechanism further includes a cam coupled to at least one articulation member, the cam being contained within the distal portion of the handle.

14. The medical device of claim 13, wherein the cam is coupled to the coupler and configured to rotate by translation of the coupler.

15. A medical device comprising:
a handle having a proximal portion that includes an actuator and a distal portion coupled to the proximal portion by a connector configured to be gripped by a user, wherein the distal portion is rotatable relative to the proximal portion by rotation of the connector;
a shaft coupled to the distal portion of the handle; and
an articulation mechanism configured to control articulation of the shaft, the articulation mechanism being disposed within the handle and extending through the connector, wherein the articulation mechanism includes a coupler translatable relative to the connector independent of rotating the connector, the coupler including:
a proximal slider coupled to the actuator; and
a distal slider coupled to at least one articulation member that extends from the handle through the shaft;
wherein the coupler is translatable relative to the connector independent of rotating the connector.

16. The medical device of claim 15, wherein a distal end of the proximal slider abuts a proximal end of the distal slider, the distal slider being rotatable relative to the proximal slider.

17. The medical device of claim 16, wherein the coupler includes a housing that surrounds the distal end of the proximal slider and the proximal end of the distal slider.

18. The medical device of claim 15, wherein the handle includes a locking mechanism to restrict rotation of the distal portion of the handle relative to the proximal portion of the handle.

19. The medical device of claim 18, wherein the locking mechanism includes a pin insertable into a plurality of apertures defined by the connector.

20. A medical device comprising:
a handle having a proximal portion and a distal portion coupled to the proximal portion by a connector configured to be gripped by a user, wherein the distal portion is rotatable relative to the proximal portion by rotation of the connector; and
an articulation mechanism disposed within the proximal portion and the distal portion of the handle and extending through the connector, wherein the articulation mechanism is configured to control articulation of a shaft coupled to the handle and includes a coupler translatable relative to the connector independent of rotating the connector;
wherein the proximal portion of the handle includes a pin insertable into a plurality of apertures defined by the connector to selectively restrict rotation of the distal portion of the handle relative to the proximal portion of the handle.
